(19) United States
(12) Patent Application Publication
(10) Pub. No.: US 2006/0173279 A1
(43) Pub. Date: Aug. 3, 2006

(54) Method for implementing a medical imaging examination procedure

(76) Inventors: Stefan Assmann, Erlangen (DE); Friedrich Fuchs, Rottenbach (DE)

Correspondence Address: SCHIFF HARDIN, LLP;PATENT DEPARTMENT, 6600 SEARS TOWER, CHICAGO, IL, 60606-6473, US

(21) Appl. No.: 11/298,032

(22) Filed: Dec. 8, 2005

(30) Foreign Application Priority Data
Dec. 8, 2004 (DE) 10 2004 059 133.4

(51) Int. Cl. A61B 5/05

(52) U.S. Cl. 600/410

(57) ABSTRACT

In a method for implementation of an imaging measurement procedure, at least three image data sets of at least one region of interest of an examination subject are obtained using different measurement parameters and/or different imaging measurement procedures. A difference image of a first and a second of the image data sets is formed and is superimposed with the third image data set. The superimposition is shown on a display medium.

### BACKGROUND OF THE INVENTION

1. Field of the Invention

The present invention concerns a method for implementation of a medical procedure involving multiple medical imaging examinations.

2. Description of the Prior Art

Through imaging examination procedures, a doctor or radiologist is able to diagnose a number of illnesses of a patient. Many illnesses require specific examination procedures in order to ensure a confident diagnosis. Imaging diagnostics are accorded a continually growing importance, particularly in the treatment of vascular illnesses but also in tumor treatment. For example, in the diagnosis and therapy of arteriosclerosis it is desirable to be able to quantitatively detect plaques occurring in the vessel system of a patient and leading to arteriosclerosis. Therapy is possible, for example, by a prescribed diet or by the use of a cholesterol-lowering drug. Conventionally, it has been possible only to monitor individual plaques over the course of time.

### SUMMARY OF THE INVENTION

An object of the present invention to provide a method that allows an overall visualization of indications of such illnesses of a patient to be obtained.

This object is achieved in accordance with the invention by a method wherein at least three image data sets of a region of interest of an examination subject are initially obtained using different measurement parameters and/or different imaging measurement procedures. A difference image between a first and a second of the image data sets is then formed. Lastly, a superimposition of the difference image and of the third image data set is generated and the superimposition is shown on a display medium. While the region of interest is, for example, completely mapped in the third image data set, by modification of the measurement parameters or by selection of a best-possible measurement procedure for the first and the second image data sets it is possible to influence the representation in the superimposition. Under best-possible imaging conditions, it is thus possible to emphasize various aspects necessary for clarification of a medical question. The imaging properties of various imaging measurement procedures and various measurement parameters are known in principle, such that those skilled in the art can find a suitable combination for a particular medical question.

Magnetic resonance tomography is used as an imaging examination procedure in an advantageous embodiment of the method. Here many possibilities of the graphical representation yield various illnesses. Specific illnesses can be particularly clearly emphasized by the optimization of the measurement parameters.

In an embodiment of the method, the first image data set is obtained using measurement parameters or an imaging measurement method selected to emphasize an indication of a particular illness. The second image data set is obtained using measurement parameters or an imaging measurement procedure such that representation of the illness indication is suppressed to the greatest possible extent. A difference image that shows only the illness indication in the region of interest is created by difference imaging. An image of the region of interest on which the illness indication is clearly emphasized appears by superimposing the difference image with the third image data set. This makes a diagnosis or an assessment of the illness easier for the treating doctor. In particular its size can be assessed in a simple manner. The respective optimal mapping for the illness to be examined can be selected by the use of various measurement procedures or measurement parameters.

In particular a representation of fatty tissue is required for identifying plaques. In an embodiment of the inventive method for this purpose, the measurement of the first image data set is implemented by means of magnetic resonance tomography without fat saturation and the measurement of the second image data set is implemented by means of magnetic resonance tomography with fat saturation. Since the images coincide other than the fat, representation after differencing only the fatty tissue of the patient remains in the difference image. The plaque content of the vessel system of the patient can be assessed in a simple manner by superimposition with the third image data set.

Since the fatty tissue of the patient generally is not limited to the vessel system, in an embodiment of the method the difference image is segmented before the superimposition. The fatty tissue not situated in the vessel system is thereby removed from the image data set. In order to also visualize plaque deposited in the vessel walls, the limit for the segmentation is approximately set outside of the vessel walls, such that the plaques situated in the vessels walls are completely retained in the image.

Magnetic resonance tomography is particularly suited for representation of the vascular system of a patient. In an embodiment of the method, the third image data set is an angiography data set or a three-dimensional angiography data set of the patient. By limiting the third image data set to the vascular system, the plaque content can be assessed in a particularly simple manner using the superimposition with the difference image.

### DESCRIPTION OF THE DRAWINGS

FIG. 1 is a flow chart of an embodiment of the inventive method.

FIG. 2 is a schematic representation of an angiography image with indicated plaques.

### DESCRIPTION OF THE PREFERRED EMBODIMENTS

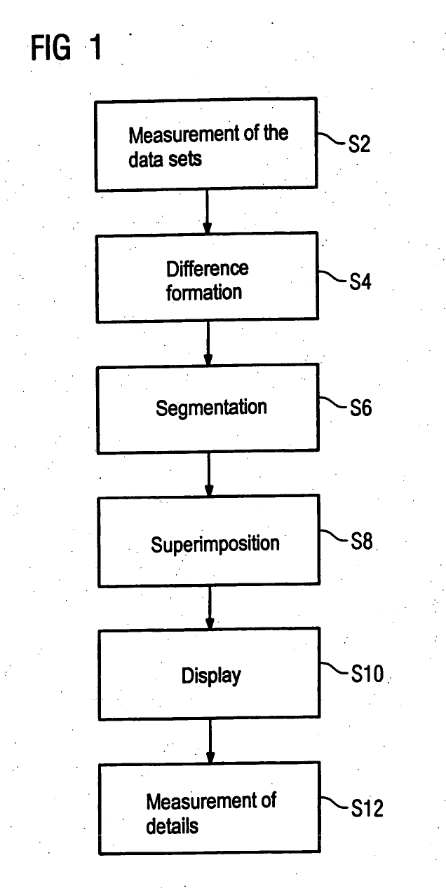

The following exemplary embodiment describes the quantification and representation of plaques in the vascular system of a patient by means of magnetic resonance examinations. According to FIG. 1, the image data sets are obtained in a first method step S2. The vascular system is thereby initially mapped according to known methods as an angiography data set or three-dimensional angiography data set in a first method step S2. The second image data set is a whole-body exposure in which, in particular, fatty tissues are made visible. The same exposure as in the second image data set is implemented as a third image data set, but this time with suppression of the fat signal. Various sequences can thereby be used, such as, for example, turbo spin echo, HASTE (Half-Fourier Acquisition Single-Shot Turbo-Spin Echo) or a proton density sequence. By means of known techniques, the fat protons are saturated by frequency-selective radio-frequency pulses to suppress the fat signal, such that it leads to a suppression of the corresponding magnetic resonance signals. The fatty tissue is not visible in the resulting image data set.

In a second method step S4, a difference image is implemented. The difference between the acquisitions with and without fat saturation is thereby formed. Only a representation of the fatty tissue of the patient from the corresponding image data sets is retained. Since, besides the plaques, additional fatty tissue generally exists in the body of the patient, the difference image is segmented in a third method step S6. The fatty tissue not situated in the vascular system is thereby removed, whereupon only the plaques belonging to the vascular system remain in the representation. A limit for the segmentation is thereby appropriately set somewhat outside of the vessel walls, such that possible plaques lying in the vessel walls are also retained. In a fourth method step S8, the difference image is superimposed with the angiography. This can be done both manually by the doctor on the monitor and automatically by means of known image processing methods. In a fifth method step S10, the superimposed image is shown on the display medium so that the doctor receives an overview of the number and distribution of the occurring plaques.

In a sixth method step S12, the doctor can subject the identified plaques to a detailed examination. All plaques or only individual plaques can thereby be selectively considered. A selection of plaques ensues with a computer mouse. They are examined in various magnetic resonance measurements, such that the doctor obtains detailed information about their size and composition. Both T1-weighted and T2-weighted measurements as well as measurements of the proton density are thereby implemented. It is furthermore possible to determine the volumes of the individual plaques with known methods. An overall sum can be calculated from the volumes of the individual plaques.

The degree of the arteriosclerosis of the patient can be quantitatively assessed from the measurement data and a corresponding therapy can be proposed. If, for example, many plaques with a high total volume are established in the body of the patient, the use of a cholesterol-lowering drug is indicated under the circumstances. Relative to known methods that could only monitor individual plaques over the course of time, the described method has the advantage of displaying the total plaque content of the vascular system of the patient. Moreover, the method is also suitable for visualization of plaques that do not yet restrict the lumen of the corresponding vessels but nevertheless represent a danger for the patient. These plaques, known as vulnerable plaques, can lead to strokes or heart attacks. For the most part these plaques do not constrict the respective vessel but are composed of fatty tissue deposited in the corresponding vessel wall. This type of plaque cannot be diagnosed by means of blood flow measurements and x-ray exposure.

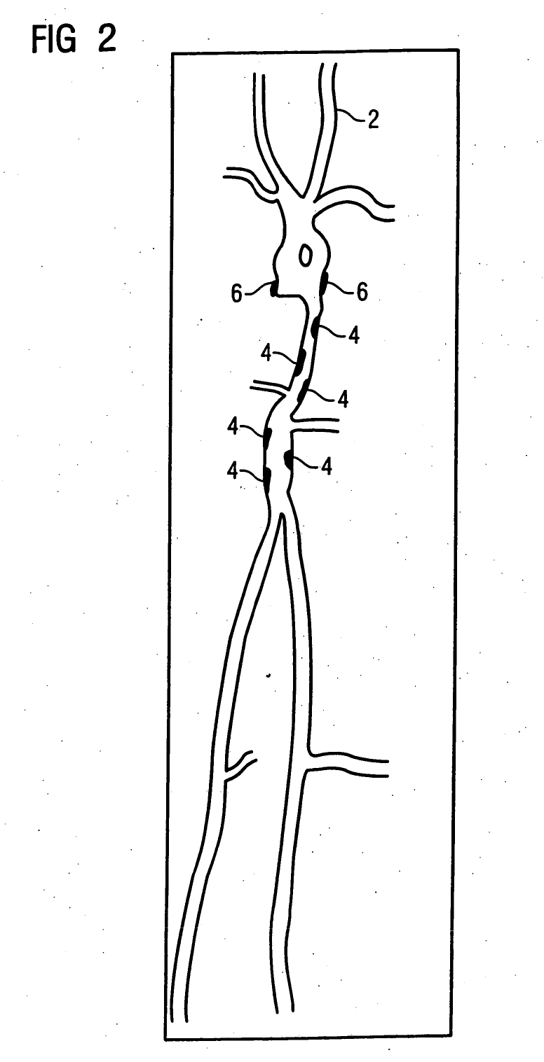

FIG. 2 shows an angiography image of the vascular system 2 of the patient in a schematic representation. It was acquired by means of magnetic resonance tomography or computer tomography. The difference image composed of two whole-body exposures with and without fat saturation is superimposed on the angiography. The difference image is segmented such that only fatty tissue associated with the vessel system is shown. The visualized plaques 4 are recognizable on the vessel walls of some vessels. A simple overview image is thus provided to the diagnosing doctor, using which overview image he can make a simple survey of the progress of the arteriosclerosis. Existing vulnerable plaques 6 can likewise be recognized.

The method can be used not only for representation of plaques but rather also in their treatment. The visualization of the plaques can thus be used for good localization in the placement of a catheter and the treatment can thereby be simplified. Plaques in the vessel walls that are otherwise not visible in an x-ray exposure can be made visible by the described method and therewith made accessible to a treatment.

The application of the method is not limited to the representation and quantification of plaques by means of magnetic resonance tomography. Different measurement procedures for acquisition of the image data sets can also be used. For example, magnetic resonance images can be superimposed with computed tomography images. The use of contrast agent offers a broad spectrum for measurement of the image data sets for the difference imaging for representation of various illnesses.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for implementing a medical procedure involving multiple medical imaging examinations of a subject, comprising the steps of:
acquiring at least three image data sets of at least one region of interest of an examination subject respectively in different examinations of the subject, selected from the group consisting of examinations using difference imaging parameters and examinations using different imaging methods;
forming a difference image of a first and a second of said image data sets;
superimposing said difference image with a third of said image data sets to obtain a superimposition image; and
visually representing said superimposition image at a display medium.

2. A method as claimed in claim 1, wherein the step of obtaining at least three image data sets comprises obtaining said at least three image data sets with a same imaging method with difference imaging parameters for respectively obtaining said at least three image data sets.

3. A method as claimed in claim 2 comprising obtaining all of said at least three image data sets by magnetic resonance tomography.

4. A method as claimed in claim 1 comprising obtaining said first and said second of said image data sets by magnetic resonance tomography, and obtaining said third of said data by computed tomography.

5. A method as claimed in claim 1 comprising selecting said examination for obtaining said first of said image data sets to emphasize indications of an illness of said examination subject, and selecting said examination for obtaining said second of said image data sets to substantially suppress said indications of said illness.

6. A method as claimed in claim 5, comprising selecting, as said examination for obtaining said first of said image data sets, magnetic resonance tomography without fat saturation and selecting, as said examination for obtaining said second of said image data sets, magnetic resonance tomography with fat saturation.

7. A method as claimed in claim 6, comprising electronically analyzing said superimposition image to identify fat deposits therein.

8. A method as claimed in claim 7, comprising electronically determining respective individual volumes of said fat deposits in said superimposition image.

9. A method as claimed in claim 8, comprising electronically summing said individual volumes of said fat deposits to form an overall sum of said fat deposits in said superimposition image.

10. A method as claimed in claim 1, comprising selecting said examination for obtaining each of said first and said second of said image data sets as a whole-body imaging method.

11. A method as claimed in claim 1, comprising electronically segmenting said difference image.

12. A method as claimed in claim 1, comprising selecting said examination for obtaining said third of said image data sets as an angiography method.

13. A method as claimed in claim 12, comprising employing three-dimensional angiography as said angiography method.
